(19) United States
(12) Patent Application Publication
(10) Pub. No.: US 2018/0263654 A1
(43) Pub. Date: Sep. 20, 2018

(54) Tissue-Removing Catheter With Abrasive Burr Having Portion Free From Abrasive Exterior Surface

(71) Applicant: Covidien LP, Mansfield, MN (US)

(72) Inventors: Bradley Steele, Plymouth, MN (US); Mark Schneider, Mound, MN (US); Thomas McPeak, Shakopee, MN (US)

(21) Appl. No.: 15/923,660

(22) Filed: Mar. 16, 2018

Related U.S. Application Data
(60) Provisional application No. 62/473,546, filed on Mar. 20, 2017.

(51) Int. Cl. A61B 17/3207

(52) U.S. Cl. CPC A61B 17/320758; A61B 2017/320004; A61B 17/320725

(57) ABSTRACT

A tissue-removing catheter generally comprises a drive shaft having opposite proximal and distal ends and configured for rotation about a longitudinal axis, and an abrasive burr operatively connected to the distal end of the drive shaft such that rotation of the drive shaft about the longitudinal axis imparts rotation to the abrasive burr. The abrasive burr includes circumferentially spaced longitudinal struts defining longitudinal slots between adjacent struts, proximal and distal hubs secured to the respective proximal and distal ends of the longitudinal struts, and an abrasive exterior surface covering an abrasive longitudinal portion of each strut extending from adjacent the proximal ends of the longitudinal struts to adjacent the distal ends of the longitudinal struts. Proximal and distal longitudinal end portions of the longitudinal struts adjacent the junctions of the longitudinal struts and the respective proximal and distal hubs are free from the abrasive exterior surface.

### CROSS-REFERENCE TO RELATED APPLICATIONS

The present application claims the benefit of U.S. Provisional Application Ser. No. 62/473,546, filed Mar. 20, 2017, the entirety of which is hereby incorporated by reference.

### FIELD OF THE DISCLOSURE

The present disclosure generally relates to a tissue-removing catheter with a rotatable burr including an abrasive exterior surface, wherein portions of the rotatable burr are free from the abrasive exterior surface.

### BACKGROUND

The patency of a body lumen may be affected by the build-up of tissue or other material in the body lumen. A variety of methods for cutting or dislodging occlusive material and removing such material from a body lumen, such as a blood vessel, have been proposed. For example, tissue-removing catheters may be used to restore the patency of a body lumen. These catheters are intended to cut or excise material from the body lumen and may employ a rotatable tissue-removing element which can be advanced into or past the occlusive material in order to cut and separate such material from the body lumen.

Although these catheters have proven very successful in restoring the patency of body lumens, problems may arise when the tissue-removing element has a smaller diameter than the occlusive tissue. If the tissue-removing element does not maintain contact with the occlusion or lumen wall, the efficacy of the tissue-removing element is reduced.

### SUMMARY

In one aspect, a tissue-removing catheter generally comprises an abrasive burr including circumferentially spaced longitudinal struts defining longitudinal slots between adjacent struts, proximal and distal hubs secured to the respective proximal and distal ends of the longitudinal struts, and an abrasive exterior surface covering an abrasive longitudinal portion of each strut extending from adjacent the proximal ends of the longitudinal struts to adjacent the distal ends of the longitudinal struts. Proximal and distal longitudinal end portions of the longitudinal struts adjacent the junctions of the longitudinal struts and the respective proximal and distal hubs are free from the abrasive exterior surface. In another aspect, a method of forming an abrasive burr for a catheter is disclosed.

Other features will be in part apparent and in part pointed out hereinafter.

### BRIEF DESCRIPTION OF THE DRAWINGS

FIG. 1 is a side elevation of an embodiment of a tissue-removing catheter, an abrasive burr of the catheter being in an initial, non-expanded configuration;

FIG. 2 is an enlarged elevational view of the tissue-removing catheter of FIG. 1;

FIG. 3 is an enlarged perspective view of the tissue-removing catheter of FIG. 1;

FIG. 4 is similar to FIG. 2, except with the abrasive burr being in an expanded configuration; and

FIG. 5 is similar to FIG. 3, except with the abrasive burr being in the expanded configuration.

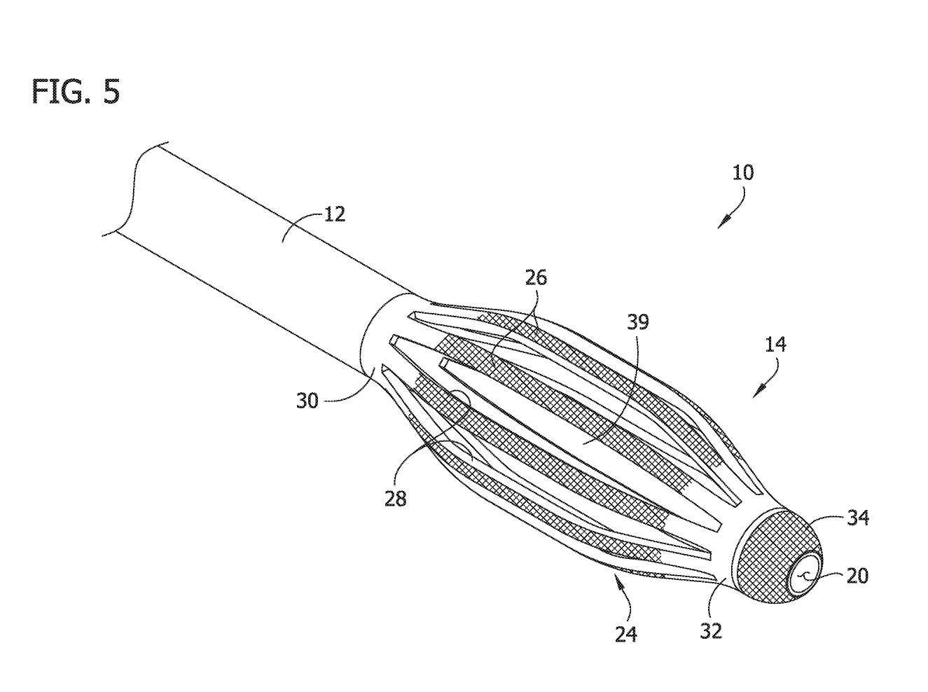

Corresponding reference characters indicate corresponding parts throughout the drawings.

### DETAILED DESCRIPTION OF THE DISCLOSURE

Referring to the drawings, an embodiment of a tissue-removing catheter for removing tissue from a body lumen is generally indicated at reference numeral 10. The illustrated tissue-removing catheter 10 is particularly suitable for removing an atheroma (e.g., plaque) from a blood vessel, although the catheter may be used to remove other occlusions from other body lumens.

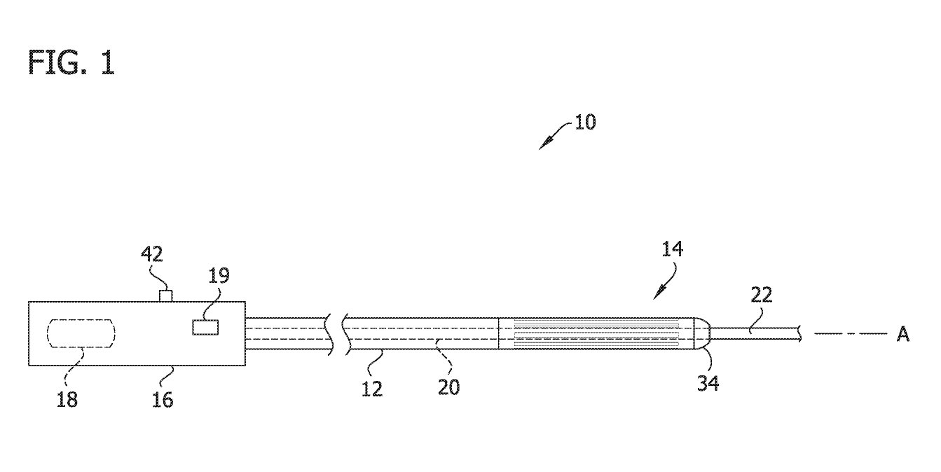

Referring to FIG. 1, the tissue-removing catheter 10 comprises a drive shaft 12, and an abrasive burr, generally indicated at 14, secured to a distal end of the drive shaft and configured to remove tissue (e.g., plaque) from a body lumen (e.g., a blood vessel). A handle 16 is operatively connected to a proximal end portion of the drive shaft 12. A motor 18 (e.g., an electric motor) or other prime mover in or otherwise operatively connected to the handle 16 is configured to rotate the drive shaft 12 and the abrasive burr 14 about an axis A (e.g., longitudinal axis of the catheter 12) to remove tissue from a lesion or other obstruction in a body lumen. The handle 16 may include a button or lever or other actuator 19 that is operatively connected to the motor 18 for actuating rotation of the drive shaft 12 and the burr 14. A guide wire lumen 20 extends longitudinally through the catheter body 12 and the abrasive burr 14 to receive a guide wire 22, which may extend distally outward from the distal end of the burr. In this way, the illustrated tissue-removing catheter 10 is configured for use as an over-wire device (i.e., over the guide wire 22). A specialty wire may be used in conjunction with the tissue-removing catheter 10 to facilitate coring through total or near total occlusions, for example. Other configurations are within the scope of the present invention, and it is understood that the guide wire 22 may be omitted within the scope of the present invention.

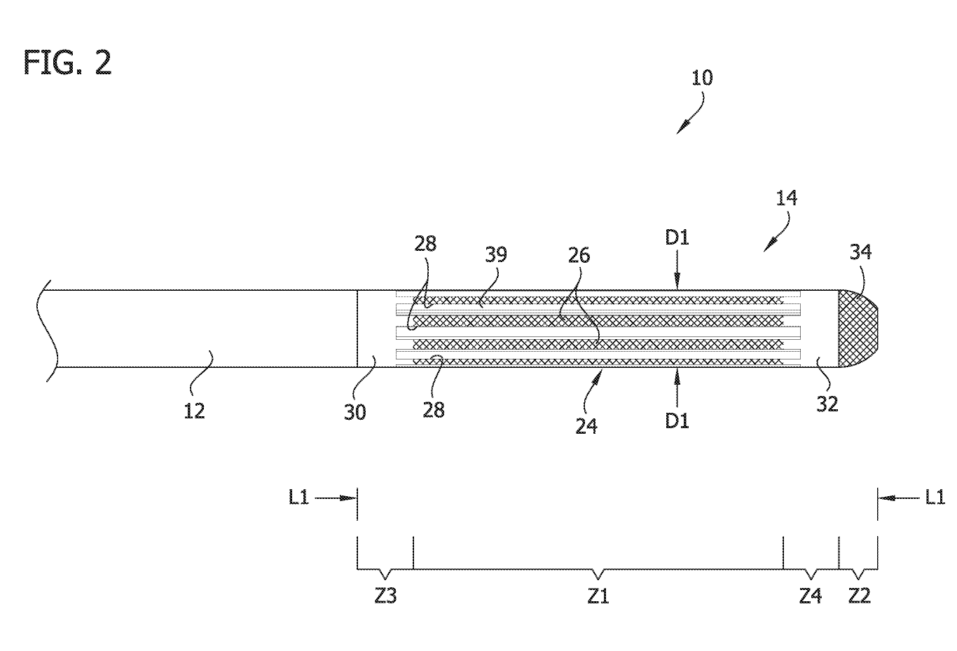

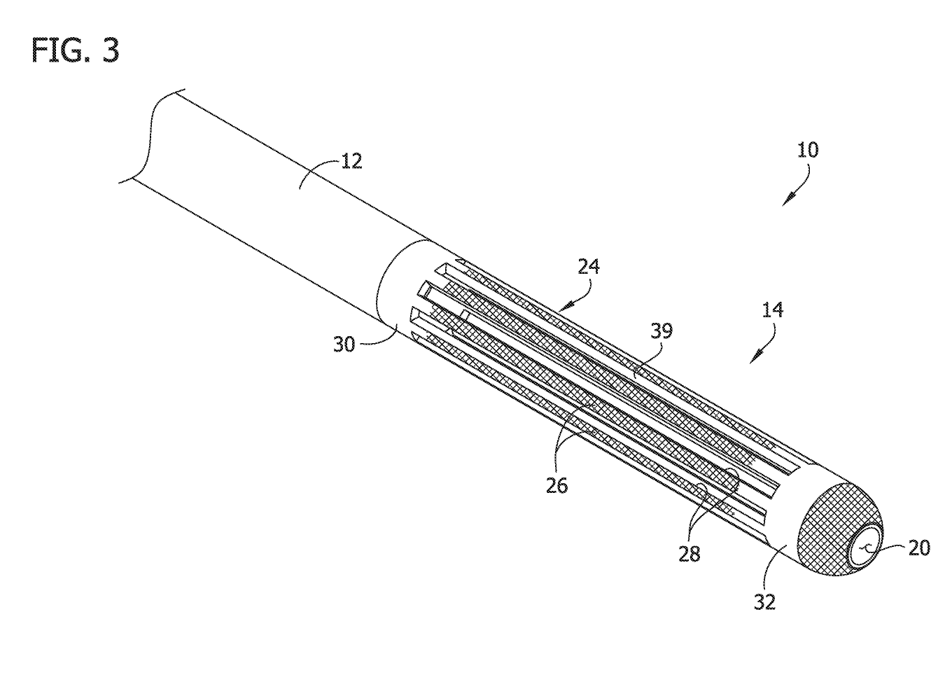

Referring to FIGS. 2 and 3, the abrasive burr 14 includes a generally tubular body, generally indicated at 24, including circumferentially spaced longitudinal struts 26 (or fingers) defining longitudinal slots 28 between adjacent struts, and proximal and distal hubs (e.g., annular hubs) 30, 32, respectively, secured to the respective proximal and distal ends of the struts. The longitudinal slots 28 permit the struts 26 to expand circumferentially relative to the proximal and distal hubs 30, 32, respectively, as described below. The abrasive burr 14 further includes a distal head 34 having a generally conical or dome-shape that tapers distally. The distal head 34 may be suitable for boring through tissue (e.g., plaque) occluding a body lumen. The tubular body 24 and the distal head 34 may be a single, one-piece component that is integrally, monolithically formed from a single piece of material. For example, the tubular body 24 and the distal head 34 may be formed from a single piece of hypotube. In other examples, the tubular body 24 and the distal head 34 may be formed separately and secured to one another in any suitable way. The tubular body 24 and the distal head 34 may be made of Nitinol, spring steel, stainless steel, or any other suitable material.

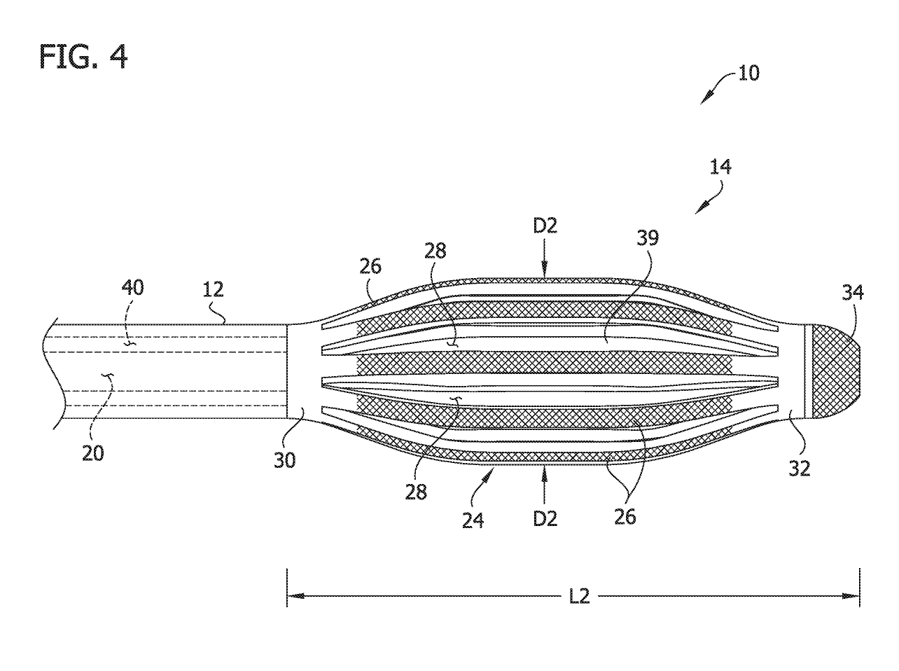

The abrasive burr 14 has an initial, non-expanded or minimum cross-sectional dimension D1 (see FIG. 2) and a first longitudinal length L1. The abrasive burr 14 is expandable circumferentially to increase the cross-sectional dimension to an expanded or maximum cross-sectional dimension D2 (FIG. 4), which is larger than the initial cross-sectional dimension D1. As illustrated, the initial cross-sectional dimension D1 is about the same as a cross-sectional dimension of the drive shaft 12, and the expanded cross-sectional dimension D2 is larger than the cross-sectional dimension of the drive shaft, although other configurations are within the scope of the present invention. In the expanded configuration, the struts 26 of the abrasive burr 14 flex or bend (broadly, deflect) outward to increase the cross-sectional dimension of the burr, which, in turn, shortens the length L1 of the burr to a second longitudinal length L2 (FIG. 4). Thus, as the cross-sectional dimension of the abrasive burr 14 increases, the longitudinal length of the abrasive burr decreases. In some embodiments, the abrasive burr 14 can be expanded and contracted to have any cross-sectional dimension in the range between the initial cross-sectional dimension D1 and a maximum expanded cross-sectional dimension D2.

The illustrated tissue-removing catheter 10 includes an expansion mechanism for use in circumferentially expanding the abrasive burr 14, more particularly, expanding the tubular body 24. In the illustrated embodiment, the catheter 10 includes a balloon 39 (broadly, an expandable member) positioned in the interior of the abrasive burr 14. The balloon 39 is inflated and deflated to control the cross-sectional dimension of the burr 14. Referring to FIG. 4, the balloon 39 is inflated by delivering fluid (e.g., liquid or gas) through a balloon lumen 40 (e.g., a balloon lumen having an annular cross section surrounding the guidewire lumen 20) in the catheter body 12. Referring to FIG. 1, the balloon lumen 40 may be fluidly connected to a fluid inlet port 42 on the handle 16 for delivering fluid from a fluid source (e.g., a syringe). In other embodiments, the balloon 39 may be inflated or deflated by a rotating pressure port (not shown) on the handle 16, and the handle may include the fluid source. As the balloon 39 is inflated, it pushes the struts 26 of the abrasive burr 14 radially outward to expand the circumference of the burr. When the balloon 39 is deflated, the burr 14 contracts toward its initial cross-sectional dimension. The burr 14 is adjustable to different cross-sectional dimensions based on the amount the balloon 39 is inflated. In one example, the balloon 39 rotates with the drive shaft 12 and the burr 14.

The expansion mechanism may be of other types and configurations for expanding the circumference of the abrasive burr 14. For example, in other embodiments the expansion mechanism may not include a balloon. Instead, the expansion mechanism may include a compressible elastomer, or other mechanism for expanding the circumference of the abrasive burr. Other suitable mechanisms are disclosed in co-pending U.S. application Ser. No. 15/189,785, filed Jun. 22, 2016, the entirety of which is hereby incorporated by reference.

The abrasive burr 14 includes an abrasive exterior surface configured to abrade tissue (e.g., plaque). The abrasive exterior surface may be formed by texturing the tubular body 24 and/or the distal head 34. For example, the tubular body 24 and the distal head 24 may be textured using a laser or may be textured in other ways to form an abrasive exterior surface. In another embodiment, the abrasive exterior surface may be formed by applying abrasive particles, such as diamond-coated or silicon carbide particles (e.g., diamond-coated grit or silicon carbide grit), to the tubular body 24 and/or the distal head 34. In these embodiments and other embodiments, the abrasive exterior surface has a suitable roughness to abrade tissue (e.g., plaque) as the abrasive burr 14 is rotated about the axis A.

In the illustrated embodiment, the abrasive exterior surface of the abrasive burr 14 covers a longitudinal portion of each strut 26 extending from adjacent the proximal ends of the struts to adjacent the distal ends of the struts, and covers at least a majority of the distal head 34. At least a majority of the length each of the struts 26 includes the abrasive exterior surface. In particular, in one embodiment about 60% to about 95% of the length of each of the struts 26 includes the abrasive exterior surface, or in another embodiment about 75% to about 90% of the length of each of the struts 26 includes the abrasive exterior surface, or in another embodiment about 80% to about 90% of the length of each of the struts 26 includes the abrasive exterior surface. In some embodiments, the entire lengths of the struts 26 include the abrasive exterior surface.

In the illustrated embodiment, proximal and distal end portions of the struts 26 (i.e., the tubular body 24) adjacent the respective proximal and distal annular hubs 30, 32 are free from the abrasive exterior surface. For example, the proximal and distal end portions of the struts 26 are not textured to form an abrasive surface and do not include abrasive particulate applied to its exterior. It is believed that because the abrasive exterior surface is not on the proximal and distal end portions of the struts 26, the proximal and distal ends of the struts are more flexible and more readily deflected relative to the proximal and distal annular hubs 30, 32 when the balloon is expanded (or other expansion mechanism is activated). Thus, the illustrated embodiment facilitates expansion and/or contraction of the abrasive burr 14.

In the illustrated embodiment, at least portions of the proximal and distal annular hubs 30, 32 are free from the abrasive exterior surface. In particular, at least portions of the proximal and distal annular hubs 30, 32 adjacent the struts 26 are free from the abrasive exterior surface. For example, the portions of the proximal and distal annular hubs 30, 32 not including the abrasive exterior surface are not textured to form an abrasive surface and do not abrasive particles applied to its exterior. The portions of the proximal and distal annular hubs 30, 32 not including the abrasive exterior surface ensure that the junction between the proximal and distal annular hubs and the respective proximal and distal end portions of the struts 26 are free from the abrasive exterior surface so that junctions are more flexible and can readily function as living hinges. In the illustrated example, the entirety of each of the proximal and distal hubs 30, 32 are free from the abrasive exterior surface. In other embodiments, portions of the proximal and distal annular hubs 30, 32 may include the abrasive exterior surface. In still other embodiments, the entirety of each of the proximal and distal annular hubs 30, 32 may include the abrasive exterior surface.

As shown in FIG. 2, the illustrated abrasive burr 14 includes zones extending circumferentially around the abrasive burr and longitudinally along the abrasive burr. Zone 1 consists of the circumferential and longitudinal portion of the tubular body 14 (e.g., the struts 26) that include the exterior abrasive surface, as described above. Zone 2 consists of the entire distal head 34 which includes the exterior abrasive surface. Zone 3 consists of the proximal annular hub 30 and the circumferential and proximal end portion of the tubular body 14 (e.g., the proximal end portions of the struts 26) which are free from the exterior abrasive surface. Zone 4 consists of the distal annular hub 32 and the circumferential and distal end portion of the tubular body 14 (e.g., the distal end portions of the struts 26) which are free from the exterior abrasive surface.

In one example, the exterior abrasive surfaces in Zones 1 and 2 have generally the same roughness. For example, the exterior abrasive surfaces in Zones 1 and 2 may have a roughness of from about 1 Ra (μm) to about 2 Ra (μm), or from about 4 Ra (μm) to about 10 Ra (μm). Where the exterior abrasive surfaces are defined by abrasive particulate applied to the burr, the Zones 1 and 2 may have a grit of from about 400 to about 600, or from about 200 to about 400

In another example, the exterior abrasive surface in Zone 2 may be coarser than the exterior abrasive surface in Zone 1. In this way, the distal head 34 is configured to more aggressively remove tissue compared to the tubular body 24. In one embodiment, the exterior abrasive surface in Zone 1 may be defined by abrasive particulate (e.g., diamond grit) and the exterior abrasive surface in Zone 2 may be defined by surface texturing (e.g., laser machining) of the tubular body 24. In this embodiment, the exterior abrasive surface in Zone 1 may have a roughness less than the roughness of the exterior abrasive surface in Zone 2, whereby the distal head 34 is more abrasive than the tubular body 24 (e.g., the struts 26). In other words, the exterior abrasive surface in Zone 2 may be coarser than the exterior abrasive surface in Zone 1. In one example, the exterior abrasive surface in Zone 1 may have a roughness from about 1 Ra (μm) to about 2 Ra (μm), and the exterior abrasive surface in Zone 2 may have a grit of from about 200 to about 400, or from about 4 Ra (μm) to about 10 Ra (μm).

In one embodiment of a tissue-removing operation, the tissue-removing catheter 10 is advanced in a body lumen over the guide wire 22 to a target site (e.g., a lesion in the body lumen). At the target site, the cross-sectional dimension of the abrasive burr 14 can be expanded initially (e.g., by using the control handle to inflate the balloon 39) or the cross-sectional dimension of the burr can initially remain in its initial, non-expanded configuration. The tissue-removing catheter 10 is activated using the control handle 16, such as by activating a control lever, button, or other device 19 to activate the motor 18. Upon activating the tissue-removing catheter 10, the drive shaft 12 rotates about the axis A, causing rotation of the abrasive burr 14. The burr 14 abrades tissue in the body lumen, thereby, in one example, expanding a diameter of an opening in the lesion in the body lumen. In one example, the distal head 34 engages the tissue to initially bore into the lesion in the body lumen and increase the diameter of the opening in the lesion. Inside the opening in the lesion, the burr 14 continues to rotate and the struts 26 engage and abrade tissue. The burr 14 can be expanded by inflating the balloon 39 for example to further abrade the tissue and increase the diameter of the opening in the lesion.

The tissue-removing catheter 10 may also be used without a guide wire. In one embodiment, the distal head 34 is used to bore through a near total or total occlusion. The tissue-removing catheter 10 may also be used without imparting rotation of the burr 14. In one embodiment, the burr 14 is used to center the tissue-removing catheter 10 in the true lumen (i.e., in the space remaining between occlusions in the body lumen). The burr 14 can be expanded circumferentially to contact the outer limits of the true lumen, thereby centering the tissue-removing catheter 10 in the true lumen. With the catheter 10 centered in the true lumen, a wire (e.g., guide wire 22) can be advanced beyond the distal head 34 to bore through tissue occluding the body lumen. In one embodiment, the burr 14 can be advanced in the initial configuration to a target site, and then expanded circumferentially at the target site to engage the occlusion to cut or tear the occlusion without rotating the tissue-removing head. In one embodiment, the burr 14 can be advanced beyond a target site, then expanded circumferentially and pulled back over the target site to remove tissue. An aspiration catheter (not shown) can be positioned near that tissue-removing head to collect debris removed by the tissue-removing head.

The tissue-removing catheter 10 facilitates creation of a larger lumen diameter. The burr 14 can increase in cross-sectional dimension to continue to enlarge an existing lumen (e.g., by using multiple passes over the same lesion), treat multiple vessels in the same patient, or treat vessels that have slight to moderate aneurysmal pockets. Because the abrasive burr 14 can decrease in cross-sectional dimension after it has been expanded, it may allow a practitioner to retrieve a stuck head or treat disease that is distal to a treatment barrier such as a stent. The variable cross-sectional dimension of the burr 14 reduces the need for using different size heads to treat multiple diameters or vessels, resulting in decreased costs. It also does not rely on centrifugal force (and thus, require a high-speed, well controlled motor) to maintain contact between the burr 14 and the lesion, but rather expands the head to contact the lesion which may result in further cost savings.

Modifications and variations of the disclosed embodiments are possible without departing from the scope of the invention defined in the appended claims.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A tissue-removing catheter comprising:
a drive shaft having opposite proximal and distal ends and configured for rotation about a longitudinal axis; and
an abrasive burr operatively connected to the distal end of the drive shaft such that rotation of the drive shaft about the longitudinal axis imparts rotation to the abrasive burr, the abrasive burr including
circumferentially spaced longitudinal struts defining longitudinal slots between adjacent struts,
proximal and distal hubs secured to the respective proximal and distal ends of the longitudinal struts, and
an abrasive exterior surface covering an abrasive longitudinal portion of each strut extending from adjacent the proximal ends of the longitudinal struts to adjacent the distal ends of the longitudinal struts,
wherein proximal and distal longitudinal end portions of the longitudinal struts adjacent the junctions of the longitudinal struts and the respective proximal and distal hubs are free from the abrasive exterior surface.

2. The tissue-removing catheter set forth in claim 1, wherein at least a distal end portion of the proximal hub adjacent the junctions of the longitudinal struts are free from the abrasive exterior surface, wherein at least a proximal end portion of the distal hub adjacent the junctions of the longitudinal struts are free from the abrasive exterior surface.

3. The tissue-removing catheter set forth in claim 2, wherein an entirety of the proximal hub is free from the abrasive exterior surface, wherein an entirety of the distal hub is free from the abrasive exterior surface.

4. The tissue-removing catheter set forth in claim 3, wherein the abrasive burr further includes a distal head disposed distal of the distal hub, wherein the abrasive exterior surface covers at least a portion of the distal head.

5. The tissue-removing catheter set forth in claim 4, wherein the distal head has a cross-sectional dimension tapering distally.

6. The tissue-removing catheter set forth in claim 5, wherein the distal head has a generally conical shape.

7. The tissue-removing catheter set forth in claim 6, wherein the abrasive exterior surface covers an entirety of the distal head.

8. The tissue-removing catheter set forth in claim 1, wherein the abrasive burr is selectively adjustable from an initial cross-sectional dimension to an expanded cross-sectional dimension larger than the initial cross-sectional dimension, in which the longitudinal struts are deflected radially outward relative to the proximal and distal hubs.

9. The tissue-removing catheter set forth in claim 8, further comprising an expansion mechanism configured to selectively adjust the abrasive burr from the initial cross-sectional dimension to the expanded cross-sectional dimension.

10. The tissue-removing catheter set forth in claim 9, wherein the expansion mechanism includes an inflatable balloon disposed inside the abrasive burr.

11. The tissue-removing catheter set forth in claim 1, wherein the abrasive exterior surface comprises at least one of textured exterior surfaces of the longitudinal struts, and an abrasive material applied to the exterior surfaces of the longitudinal struts.

12. The tissue-removing catheter set forth in claim 11, wherein the abrasive exterior surface comprises the abrasive material.

13. The tissue-removing catheter set forth in claim 12, wherein the abrasive material is diamond-coated grit.

14. The tissue-removing catheter set forth in claim 11, wherein the abrasive exterior surface comprises the textured exterior surfaces of the longitudinal struts.

15. The tissue-removing catheter set forth in claim 14, wherein the textured exterior surfaces of the longitudinal struts are formed by laser machining.

16. The tissue-removing catheter set forth in claim 1, wherein the abrasive burr further includes a distal head disposed distal of the distal hub, wherein the abrasive exterior surface covers at least a portion of the distal head.

17. The tissue-removing catheter set forth in claim 16, wherein the abrasive exterior surface on the distal head has a roughness greater than the roughness of the abrasive exterior surface on the longitudinal struts.

18. The tissue-removing catheter set forth in claim 17, wherein the abrasive exterior surface on the distal head comprises diamond-coated grit, wherein the abrasive exterior surface on the longitudinal struts comprises textured exterior surfaces of the longitudinal struts.

19. A method of forming an abrasive burr for a catheter, the method comprising:
forming circumferentially spaced longitudinal struts defining longitudinal slots between adjacent struts,
forming proximal and distal hubs secured to the respective proximal and distal ends of the longitudinal struts, and
forming an abrasive exterior surface covering an abrasive longitudinal portion of each strut extending from adjacent the proximal ends of the longitudinal struts to adjacent the distal ends of the longitudinal struts, wherein proximal and distal longitudinal end portions of the longitudinal struts adjacent the junctions of the longitudinal struts and the respective proximal and distal hubs are free from the abrasive exterior surface.

20. The method of forming an abrasive burr for a catheter set forth in claim 19, wherein the method further comprising:
forming a distal head connected to and disposed distal of the distal hub; and
forming a second abrasive exterior surface covering at least a portion of the distal head.
